(19) United States
(12) Patent Application Publication
(10) Pub. No.: US 2005/0277625 A1
(43) Pub. Date: Dec. 15, 2005

(54) Estriol and estetrol prodrugs

(76) Inventors: Ralf Wyrwa, Rothenstein (DE); Sven Ring, Jena (DE); Peter Droescher, Weimar (DE); Alexander Hillisch, Jena (DE); Walter Elger, Berlin (DE); Birgitt Schneider, Jena (DE); Gudrun Reddersen, Jena (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/134,618

(22) Filed: May 23, 2005

Related U.S. Application Data
(60) Provisional application No. 60/572,972, filed on May 21, 2004.

(30) Foreign Application Priority Data
May 21, 2004 (DE) 102004025985.2

(51) Int. Cl. A61K 31/56; C07J 1/00

(52) U.S. Cl. 514/182; 552/626

(57) ABSTRACT

This invention provides estriol and estetrol prodrugs of general formula (I), in which group Y is bonded to the steroid 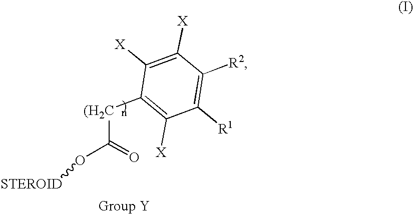 process for their production, pharmaceutical compositions that contain these compounds, as well as their use for the production of pharmaceutical agents with estrogenic action.

This application claims the benefit of the filing date of U.S. Provisional Application Ser. No. 60/572,972 filed May 21, 2004 which is incorporated by reference herein.

The invention relates to estriol and estetrol prodrugs of general formula I, 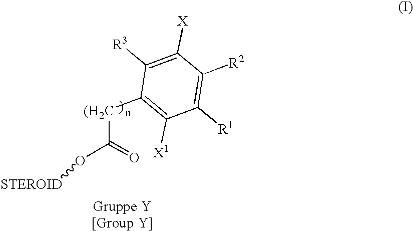
process for their production, pharmaceutical compositions that contain this compound, and their use for the production of pharmaceutical agents with estrogenic action.

Estrogens control many functions on the cellular plane. They play a central role in the functions of the genital organs in both sexes. Moreover, estrogenic actions in the central nervous system play an important role for the organization of the CNS (central nervous system), the modulation of behavior, and the monitoring of the gonadotropin secretion of the pituitary gland. Also, other pituitary hormones are modulated by estrogens.

Their action is mediated by at least two known receptors, ERα and ERβ, which are expressed in very different concentrations in the entire organism, i.e., even outside of the genital organs⁽¹⁾.

In women, the estrogens that are secreted by the ovary dominate in the organism, whereby the focus is placed entirely on the secretion of estradiol. In pregnancy, the placenta forms large amounts of estrogen, whereby very large amounts of estriol are secreted in particular in the later phases of pregnancy⁽²⁾.

In men, estrogens are produced predominantly “peripherally” by the aromatization of testosterone or adrenal androgens in various effector organs, such as the CNS, bone, fatty tissue, or the intestine. This organ-selective adaptation allows physiological estrogen effects in men in the case of very low estradiol levels in the blood.

The loss of estrogens or their disrupted formation (defect or inhibition of the aromatase) is accompanied by disruptions of a broad spectrum of organ and metabolic functions⁽³⁾. This spectrum comprises disruptions or loss of reproductive function, disruptions in carbohydrate metabolism and lipometabolism, but primarily also disorders of the skeletal system⁽⁴⁾. In youths, abnormalities in linear growth predominate; in later age, changes in bone density predominate. Loss of bone mass (osteoporosis) is the most dangerous result of estrogen deficiency after linear growth has ended, since it is accompanied by an elevated brittleness of bone and frequently is the cause of disability in older age⁽⁵⁾.

In women, estrogens have important circulatory functions. In premenopausal terms, their actions are an essential factor for the lower risk in females with respect to cardiovascular morbidity and mortality rates. The loss of estrogens results in unfavorable changes in lipoproteins and in the endothelial functions with respect to the distending of vessels and the prevention of clotting⁽⁶⁾.

Urogenital functions are also estrogen-modulated. The build-up and the regeneration of epithelial cells and muscles in the urethra and bladder are positively influenced by estrogens⁽⁷⁾.

The use of estrogens has a solid place in gynecological therapy. This comprises the use in hormonal contraceptive agents and in various forms of estrogen substitution therapy. Hormonal contraceptive agents inhibit the ovulation and thus the ovarian secretion of estrogens and progesterone. At the same time, they are substituted by endogenous sex hormones in the genital tract and in the entire organism. Their most important function in the uterus is in this connection the stabilization of the mucous membrane of the uterus to achieve a good menstrual control.

In therapeutic applications, estrogens can be supplied orally and parenterally. For oral therapy, natural estrogens, such as estradiol or derivatives thereof, e.g., estradiol valerate, are used. Estrogen mixtures that are obtained from the urine of pregnant mares, the so-called conjugated estrogens, play a major role in oral estrogen therapy. The latter represent sulfates, i.a., from estrone and from estrogens that are typical of the horse (equilin and equilenin). After being taken up in the organism, they are hydrolytically cleaved; in this connection, the therapeutically relevant “mother estrogens” are released. These estrogens dominate all forms of menopausal substitution therapy. Despite certain oral bioavailability, however, they do not play any role for hormonal contraception. The essential reason for this is an inadequate control of uterine bleeding by natural estrogens and derivatives thereof (esters) that correspond to the prior art.

The hormonal contraception is completely dominated by ethinyl estradiol. The basis for the poor menstrual control by estradiol versus ethinyl estradiol is the metabolization of the estradiol in the endometrium with the simultaneous application of a gestagen. The enzyme 17β-hydroxy steroid dehydrogenase (17βHSD) is induced in the human endometrium by progesterone and gestagens⁽⁸⁾. Estradiol converts the latter into the much weaker estrogen estrone. In the case of ethinyl estradiol, a corresponding oxidation step is not possible. This also applies for the substances according to the invention. Estriol and estetrol are not metabolized by the 17βHSD in the endometrium.

An essential feature of the conventional estrogen therapy is the necessity for very much higher doses than a parenteral application would require. This is the reason why oral estrogen treatment or therapy has other metabolic effects than an equivalent parenteral treatment or therapy, even though natural estrogens have indeed been used⁽⁹⁾. The ethinyl estradiol, however, has especially strong hepatic estrogeneity, since it is inactivated only with a time lag in the liver⁽¹⁰⁾. Estrogen-sensitive hepatic functions pertain to the renin-angiotensinogen aldosterone system and thus blood pressure regulation⁽¹¹⁾. For the muscles and the skeletal system, a reduction of the hepatic IGF-I synthesis is unfavorable⁽¹²⁾. There are many other hepatic functions that are also deflected by estrogens.

In the case of estradiol, often 40× higher doses are used for an oral therapy than in a therapeutically equivalent transdermal therapy. The high dose that is necessary for oral therapy accordingly has the drawback of strong estrogen effects in the liver, in which after being resorbed, the total quantity administered first goes with the portal blood and whereby most of the amount of substance is metabolized⁽¹³⁾.

Steroidally active compounds that are bonded to erythrocytes via the group —SO₂NR¹R² and that accumulate there are known from DE 100 27 887.6 A1. The concentration ratio of the compounds between erythrocytes and plasma is 10-1000, preferably 30-1000, so that it is possible to speak of a depot formation in the erythrocytes. By the strong binding of the compounds to the erythrocytes, metabolization while passing through the liver is avoided. Disadvantageously, despite a reduced metabolization with the indicated dosages, no therapy-relevant active ingredient levels are provided.

It is the object of the invention to prepare new steroidal compounds with estrogenic action that are orally available and, in comparison to the prior art, also ensure a therapy-relevant active ingredient level even at a lower dosage.

This object is achieved by estriol and estetrol prodrugs of general formula (I), in which group Y is bonded to the steroid that is to be released, 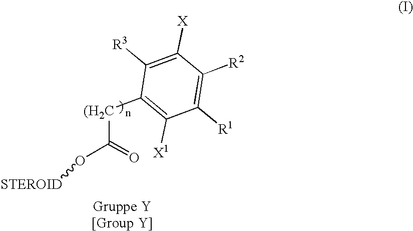
in which n is a number 0-4,
R¹ is a radical —SO₂NH₂ or —NHSO₂NH₂,
   whereby R², R³ and X, X¹ stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
   whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and R²⁰ in addition can mean a hydrogen, or
R² is a radical —SO₂NH₂ or —NHSO₂NH₂,
   whereby R², R³ and X, X¹ stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
   whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and R²⁰ in addition can mean a hydrogen, or
R³ is a radical —SO₂NH₂ or —NHSO₂NH₂,
   whereby R², R³ and X, X¹ stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
   whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and R²⁰ in addition can mean a hydrogen, and
STEROID stands for a steroidal ABCD-ring system of general partial formulas II A to II D 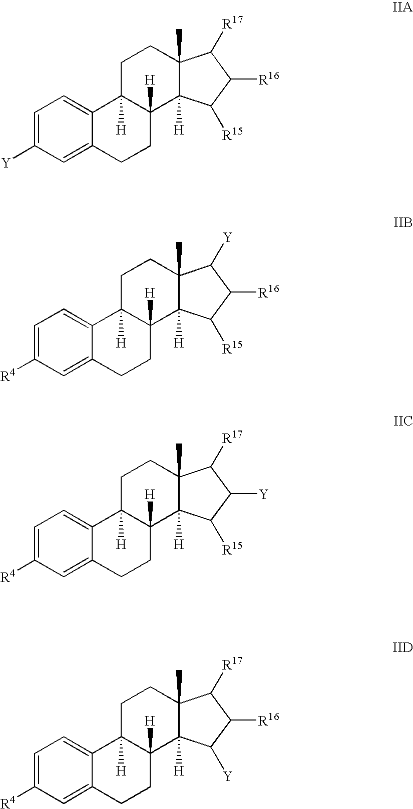
   whereby
R⁴, R¹⁶, R¹⁷ can represent a hydroxy group, a tri(C₁-C₆-alkyl)silyloxy group, a group OC(O)—R²⁰; a C₂₋₅-heterocycloalkyloxy group, or a group Y and
R¹⁵ can represent a hydrogen atom, a hydroxy group, a tri(C₁-C₆-alkyl)silyloxy group, a group OC(O)—R²⁰, a C₂₋₅-heterocycloalkyloxy group, or a group Y, and
their pharmaceutically acceptable salts.

The compounds according to the invention have estrogenic activity.

In the context of this invention, “C₁₋₅-alkyl group” is defined as a branched or straight-chain alkyl radical with 1 to 5 carbon atoms, which can be substituted by, for example, halogen atoms, a hydroxy group, or a nitrile group. As examples, a methyl, ethyl, n-propyl, i-propyl, n-butyl, i-butyl, tert-butyl or n-pentyl group can be mentioned.

According to the invention, the above-mentioned “C₃₋₈-cycloalkyl group” means a monocyclic or bicyclic group, which can be substituted by, for example, halogen atoms, a hydroxy group, or a nitrile group, such as, for example, a cyclopropyl, cyclobutyl, cyclopentyl, cyclohexyl or a hydroxycyclohexyl group.

In the context of this application, the term “aryl group” is defined as a substituted or unsubstituted aryl radical with 6 to 15 carbon atoms, for example, a phenyl group, a substituted phenyl group, such as a halophenyl group, or a nitrophenyl group, or a naphthyl group.

In the context of this application, the term “C₁₋₄-alkylene aryl group” is defined as a disubstituted alkyl radical that is substituted at least with an aryl radical. Both radicals together have 7 to 15 carbon atoms, whereby the group can carry additional substituents, such as, for example, a halogen atom. Examples are a benzyl group or a halobenzyl group.

In the context of this application, the term “C₁₋₄-alkylene-C₃₋₈-cycloalkyl group” is defined as a disubstituted alkyl radical that is substituted at least with a C₃₋₈-cycloalkyl radical. Both radicals together exhibit 7 to 15 carbon atoms, whereby the group can carry additional substituents, such as, for example, a halogen atom. Examples are a cyclopentylethyl, cyclohexylmethyl or cyclohexylethyl group

In the context of this application, the term “C₃₋₈-cycloalkylene-C₁₋₄-alkyl group” is defined as a disubstituted C₃₋₈-cycloalkylene radical that is substituted at least with a C₁₋₄-alkyl radical. Both radicals together exhibit 7 to 15 carbon atoms, whereby the group can carry additional substituents, such as, for example, a halogen atom. Examples are a propylcyclohexyl group or a butylcyclohexyl group.

According to this invention, the term “CₚF₂ₚ₊₁-group” is defined as a perfluorinated alkyl radical, such as, for example, a trifluoromethyl radical and a pentafluoroethyl radical.

The term tri(C₁₋₆-alkyl)silyloxy group is defined as, for example, a trimethylsilyloxy group, a triisopropylsilyloxy group, a thexyldimethylsilyloxy group or a tert butyldimethylsilyloxy group.

Within the scope of the invention, the term “C₂₋₅-heterocycloalkyloxy group” is defined as a C₂₋₅-heterocycloalkyloxy group with a nitrogen atom or an oxygen atom as a heteroatom, whereby the binding of the C₁₋₅-heterocycloalkyloxy group is carried out via the oxygen atom in 2-, 3- or 4-position. An example of this is the perhydropyranoxy group.

Within the scope of this invention, the term “halogen atom” is defined as a fluorine, chlorine, bromine or iodine atom; a fluorine, chlorine or bromine atom is preferred.

The number “n” is preferably 0, 1 and 2, and especially preferably 0.

STEROID preferably stands for a steroidal ABCD-ring system of general partial formulas II B and II C.

It is preferred that R¹ represent a radical —SO₂NH₂ or —NHSO₂NH₂, whereby the radical —SO₂NH₂ is especially preferred. The above-mentioned radicals thus are found in m-position of group Y in relation to the ester group, via which group Y is bonded to the steroid.
R¹ preferably means a group —SO₂NH₂, whereby R², R³, X¹ and X are preferably a hydrogen atom, a fluorine atom, a chlorine atom, a hydroxy group or a methoxy group, or
R² preferably means a group —SO₂NH₂, whereby R¹, R³, X¹ and X preferably are a hydrogen atom, a fluorine atom, a chlorine atom, a hydroxy group or a methoxy group, or
R³ preferably means a group —SO₂NH₂, whereby R¹, R², X¹ and X preferably are a hydrogen atom, a fluorine atom, a chlorine atom, a hydroxy group or a methoxy group.
R⁴, R¹⁶ and R¹⁷ are preferably in each case and independently of one another in a hydroxy group, a trimethylsilyloxy group, a tert-butyldimethylsilyloxy group, a benzoate group, an acetate group, a propionate group, a valerate group, a butciclate group or a cyclopentylpropionate group, whereby the hydroxy group is especially preferred.
R¹⁵ is preferably a hydrogen atom.

Radicals R¹⁵, R¹⁶, and R¹⁷ can be arranged both in α-position and in β-position.

Especially preferred compounds or estriol or estetrol prodrugs are cited below:
1) 3,16α-Dihydroxyestra-1,3,5(10)-trien-17β-yl 3′-sulfamoylbenzoate (7),
2) 3,16α-Dihydroxyestra-1,3,5(10)-trien-17β-yl 4′-sulfamoylbenzoate (1),
3) 3-tert.-butyldimethylsilyloxy-16α-hydroxyestra-1,3,5(10)-trien-17β-yl 3′-sulfamoylbenzoate,
4) 3-tert.-butyldimethylsilyloxy-16α-hydroxyestra-1,3,5(10)-trien-17β-yl 4′-sulfamoylbenzoate,
5) 3,17β-Dihydroxyestra-1,3,5(10)-trien-16α-yl 3′-sulfamoylbenzoate (10),
6) 3,17β-Dihydroxyestra-1,3,5(10)-trien-16α-yl 4′-sulfamoylbenzoate (4),
7) 3-tert.-butyldimethylsilyloxy-17β-hydroxyestra-1,3,5(10)-trien-16α-yl 3′-sulfamoylbenzoate,
8) 3-tert.-butyldimethylsilyloxy-17β-hydroxyestra-1,3,5(1)-trien-16α-yl 4′-sulfamoylbenzoate,
9) 3,16α-Dihydroxyestra-1,3,5(10)-trien-17β-yl 2′-chloro-5′-sulfamoylbenzoate,
10) 16α, 17β-Dihydroxyestra-1,3,5(10)-trien-3-yl 4′-sulfamoylbenzoate (13),
11) 3,15α, 16α-Trihydroxyestra-1,3,5(10)-trien-17β-yl 3′-sulfamoylbenzoate,
12) 3,15α, 16α-Trihydroxyestra-1,3,5(10)-trien-17β-yl 4′-sulfamoylbenzoate,
13) 3,15α, 17β-Trihydroxyestra-1,3,5(10)-trien-16α-yl 3′-sulfamoylbenzoate,
14) 3,15α, 17β-Trihydroxyestra-1,3,5(10)-trien-16α-yl 4′-sulfamoylbenzoate,
15) 3,16α, 17≢-Trihydroxyestra-1,3,5(10)-trien-15α-yl 3′-sulfamoylbenzoate,
16) 3,16α, 17β-Trihydroxyestra-1,3,5(10)-trien-15α-yl 4′-sulfamoylbenzoate,
17) 15α, 16α, 17β-Trihydroxyestra-1,3,5(10)-trien-3yl 3′-sulfamoylbenzoate and
18) 15α, 16α, 17β-Trihydroxyestra-1,3,5(10)-trien-3yl 4′-sulfamoylbenzoate.

For the formation of pharmaceutically acceptable salts of the compounds of general formula I according to the invention, i.a., hydrochloric acid, hydrobromic acid, sulfuric acid and phosphoric acid are considered as inorganic acids, and, i.a., acetic acid, propionic acid, maleic acid, fumaric acid, succinic acid, benzoic acid, ascorbic acid, oxalic acid, salicylic acid, tartaric acid, citric acid, lactic acid, malic acid, mandelic acid, cinnamic acid and methanesulfonic acid are considered as organic acids.

The compounds according to the invention have estrogenic activity in the case of oral administration, whereby hepatic effects, other than in the case of conventional estrogens, are avoided to a very large extent. The compounds according to the invention do not themselves bind to the estrogen receptor, but rather the therapeutically relevant steroid is released from the latter by ester cleavage.

In-Vivo Tests:

Test I

Surprisingly and unexpectedly, a higher estrogenic activity and oral bioavailability are achieved in in-vivo experiments in rats in the case of oral administration for m-substituted compound 7 than for m-substituted compound 10 or p-substituted compound 1.

Table 1 illustrates the estrogenic activity in the case of oral administration and liver toxicity (hepatic effects) based on data of selected compounds in in-vivo tests with rats.

Principle of the Test and Test Description:

Adult Wistar rats are ovariectomized and are brought into the test after a 14-day waiting period. The animals are then treated for over three days (days 1-3) and then sacrificed (day 4). Then, the organ weights are determined and estrogen-modulated factors of hepatic secretion in serum are determined, which in the rat are the increase of angiotensinogen and the drop in total cholesterol and HDL cholesterol.

If the increase of the uterus weight is taken as an indicator of systemic estrogenic effect, it is clear that, even at very high dosages, estriol did not bring about a doubling of the uterus weight. With the substances in Table 1 according to the invention, a fraction of the dose that was tested for estriol is sufficient to induce a corresponding degree of uterus growth. Unlike in the uterus, estrogen effects in the liver manifest very clearly with the treatment of non-derivatized estriol. In absolute terms, hepatic estrogeneity is not overwhelmingly reduced in comparison to estriol with the substances according to the invention, but therapeutically relevant effects can be achieved in the uterus in relation to the much lower dose.

TABLE 1
Comparison of Estrogenic Activity and Hepatic Effects in
Ovariectomized (OVX) Rats

| | | | Total | HDL |
|---|---|---|---|---|
| | Uterus | Angiotensin 1 | Cholesterol | Cholesterol |
| | Weight | ED50 [symbol] | ED50 [symbol] | ED50 [symbol] |
| | UVD | (μg/T/T) | (μg/T/T) | (μg/T/T) |
| Compound | (μg/T/T) | rel. hep. Akt. | rel. hep. Akt. | rel. hep. Akt. |
| Estriol | >1000 | 7.6 | 79 | 110.7 |
| | | <0.008 | <0.08 | <0.1 |
| 1 | 162.6 | 17.4 | 39.9 | 45.9 |
| | | 0.12 | 0.25 | 0.28 |
| 7 | 137.5 | 8.9 | 4.8 | 6.0 |
| | | 0.06 | 0.03 | 0.04 |
| 10 | 378.2 | 5.7 | 10.7 | 18.6 |
| | | 0.015 | 0.028 | 0.49 |

UVD = Uterus Doubling Dose vs. OVX;
ED 50 [symbol] , [symbol] = dose, which produces an increase or drop by 50% vs. OVX control;
rel. hep. Akt. = relative hepatic activity vs. uterotropic activity

The action of estriol in the livers of rats that is stronger in comparison to estradiol reflects a reduced oxidation of the 17β-OH function of this estrogen by the presence of the 16α-OH group and inactivation in rats. An undiminished estrogenic effect in the uterus in comparison to other natural estrogens (estradiol) in particular has a negative effect if the 17β-hydroxy steroid dehydrogenase in the uterus is elevated by gestagens, and estradiol is very quickly degraded in the mucous membrane of the uterus.

Test II

Surprisingly enough, it was now possible to determine that it is possible with estriol and the corresponding compounds according to the invention to prevent the weakening of estrogenic effects in the uterus by a gestagen.

Principle of the Test and Test Description:

Adult guinea pigs were ovariectomized and treated for 2 weeks after this operation over 7 days with estradiol or estriol over a large dose range. The selected dosages of the estrogens were administered a) alone and b) in combination with a full gestagenically active dose of 3.0 mg of progesterone per animal per day by subcutaneous injection in oily solution. The effects of this treatment on the genital organs was raised on the 8^{th} test day.

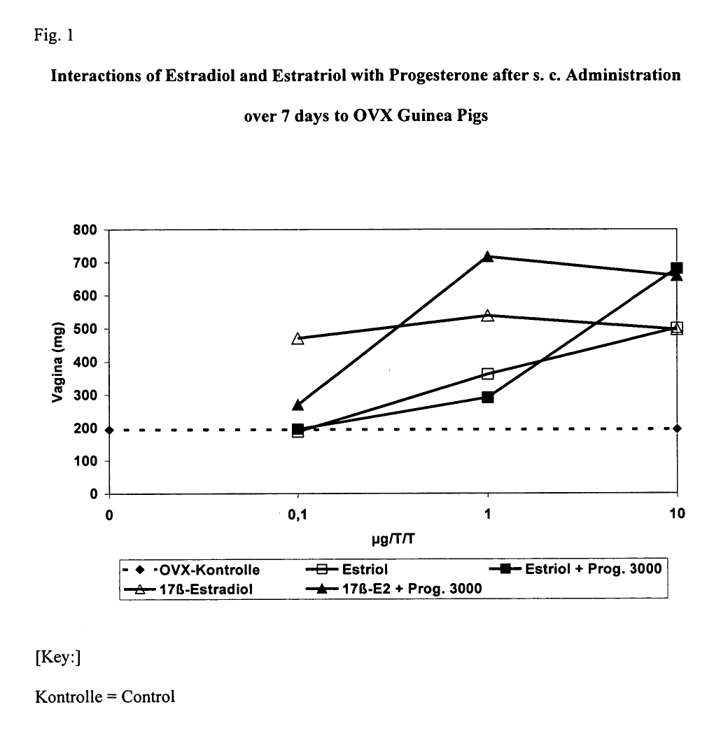

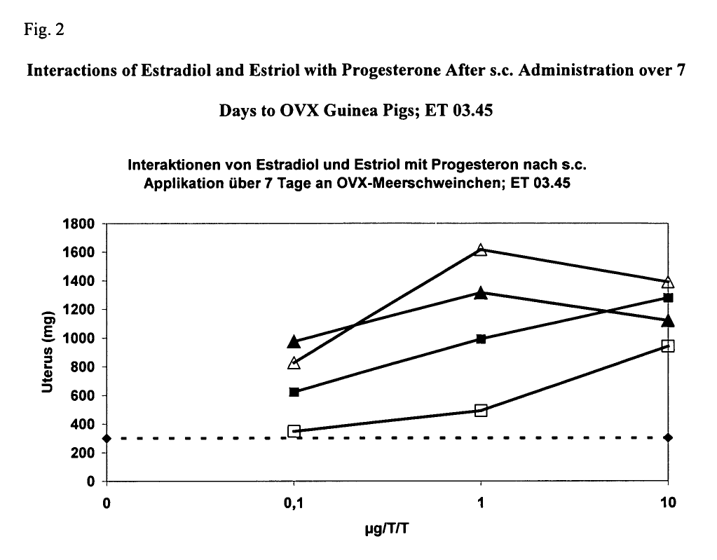

In FIGS. 1 and 2, different interactions of estradiol and estriol with progesterone in the vagina and in the uterus of ovariectomized guinea pigs are shown, treatment days 1-7, autopsy day 8.

In this case, progesterone weakens the uterotropic actions of estradiol, while the corresponding actions of estriol are enhanced.

In the case of estradiol and estriol, opposite interactions are raised. This is thus also surprising since estriol in general is seen as essentially weaker estrogen. Progesterone weakens the uterotropic actions of estradiol, while the corresponding actions of estriol in all dosages of estriol that were tested are clearly enhanced. The observed interaction is specific to the uterus. The increase in weight of the vagina by estriol is not enhanced or is not enhanced to the same extent by progesterone; inhibitory effects of progesterone predominate.

For this reason, the compounds according to the invention are superior to the estradiol with respect to preventing breakthrough and intracyclic menstrual bleeding under simultaneous treatment with a gestagen. Since in humans, estriol is very quickly inactivated in the liver by conjugation (glucuronidation, sulfation), in humans—unlike in rats—no deviations of estrogen-modulated liver functions occur even under very high oral doses of estriol. Substances according to the invention accordingly can be used in humans, unlike as estradiol and ethinyl estradiol in doses that act fully uterotropically, without undesirable effects in the liver having to be tolerated.

In-Vitro Tests:

Test I

Studies with respect to bonds of m-substituted compounds 7 and 10 to erythrocytes were also surprising. For m-substituted compounds, only very weak bonds were detected.

In this case, however, it was unexpected that the m-substituted compounds, despite lower binding strength to erythrocytes, have varying oral availability and estrogeneity. The data in Table 2 are to illustrate the binding to erythrocytes of selected compounds according to Formula I.

TABLE 2
Binding of Selected Compounds to Erythrocytes

| Compound | RBA to Erythrocytes |
|---|---|
| Estradiol-3-sulfamate | 100 |
| 7 | 0.5 |
| 4 | 2.6 |
| 10 | 0.4 |

Principle of the Test and Test Description:

The SO₂—NH₂ group of the substances according to the invention can result in a concentration in erythrocytes by binding to carboanhydrases. The displacement of estradiol-3-sulfamate from the erythrocyte bond by test substances is measured.

Test preparation: Human blood is mixed with a mixture that consists of ¹⁴C-labeled and unlabeled estradiol sulfamate. The erythrocytes are saturated at the selected working point, and the distribution in plasma/erythrocytes is 40:60. A second blood sample is mixed with a mixture that consists of ¹⁴C-labeled estradiol sulfamate and unlabeled test substance. The relative binding affinity is calculated from the proportion of ¹⁴C-labeled estradiol sulfamate in plasma: higher proportion=strong displacement of ¹⁴C-estradiol sulfamate from the erythrocytes by the test substance=high binding affinity.

Test II

Surprisingly enough, in all cases a bond to the carboanhydrase (CA I) that is found in the erythrocytes nevertheless could be shown (Table 3). It is to be expected, therefore, that the compounds according to the invention also have therapeutically relevant effects as carboanhydrase inhibitors. The bond to erythrocytes that is induced by the high affinity to carboanhydrases is important for properties as estrogen, however. This bond is essential for a reduced extraction of the orally administered substance in the first liver passage. High or low affinity to the erythrocytic carboanhydrases, faster or delayed release from this depot, and subsequent hydrolysis determine the therapeutic usability of the substances according to the invention. The compounds according to the invention thus open up the possibility that higher shorter-term or uniformly low and longer-lasting hormone levels can be achieved with the same absolute amount of substance administered. As a result, active strengths and durations of action can be varied. In this respect, a therapy that is matched to the individual organism is made possible.

TABLE 3
IC₅₀ Inhibiting Values of Human Carboanhydrase I

| | CAI |
|---|---|
| Inhibitor | IC50 (nM) |
| Estradiol-3-sulfamate | 157 ± 10.6 |
| 10 | 450 |
| 7 | 600 |
| 1 | 200 |
| Acetazolamide | 1200 |
| (of known CA inhibitors) | 1900⁽¹⁴⁾ |

Principle of the Test and Test Description:

Carboanhydrases catalyze the CO₂ hydration.

Test preparation: A constant CO₂ stream is directed by a buffer that was mixed with carboanhydrase I. The time that is required to reduce the pH within defined limits is a measuring parameter. This parameter reflects the formation of H₂CO₃ in the medium. IC₅₀ inhibiting values are determined by test substances being pipetted into the test preparation. In the concentration areas that are examined, the test substances cause no inhibition to complete inhibition of the above-mentioned enzymes.

These test results open up many possible applications in the compounds of general formula (I) according to the invention for birth control and hormone replacement therapy (HRT) in women or the treatment of hormonally induced diseases in men and women or else for treatment of carcinomas, such as, e.g., prostate cancer.

The substances according to the invention are suitable to achieve menstrual control with natural hormones and simultaneously to avert dangerous hepatic estrogenic effects, as they occur typically and especially strongly under ethinyl estradiol and other estrogens that are not natural.

Preventing corresponding first-pass interactions is another purpose of the substances according to the invention. The latter can pass through the liver for the most part and also in inactive form. They can be much lower-dosed to achieve a desired systemic estrogenic effect than their “mother estrogens” and have correspondingly fewer undesirable effects in the liver.

The substances according to the invention are preferably used for oral therapy. Compared to their mother estrogens, the compounds according to the invention have a clearly increased oral bioavailability, an increased systemic, but reduced hepatic estrogeneity.

By this dissociation of desirable and undesirable hormonal effects, simultaneously more therapeutically effective and, in comparison to the prior art, more compatible pharmaceutical agents are made possible.

In the case of oral therapy with natural estrogens (estradiol, estradiol valerate, estrone sulfate, conjugated estrogens), but also in that with estradiol sulfamate, high levels of estrone predominate in the blood. This is an important deviation from the menstrual ratios, where the concentrations of estradiol in the blood are higher than those of estrone. This is therefore disadvantageous, since estrone is a much less effective estrogen than estradiol.

A special advantage of the compounds according to the invention in comparison to the prior art is therefore the release according to the invention of the mother estrogen that is not oxidized in each case, preferably that of estriol and estetrol. This contributes to the desirable high systemic estrogeneity of the substances according to the invention. In addition, an inadequate metabolization of these estrogens in the uterus, in the context of an organ-selective enhanced estrogeneity, has proven advantageous.

The compounds according to the invention have estrogenic activity in the case of parenteral and oral administration, whereby hepatic effects are reduced in comparison to equipotent dosages of estriol relative to the action on the uterus. The oxidation in 17-position to weak estrone derivatives is avoided. In this respect, some desirable estrogenic actions in those effector organs for estrogens are absolutely or relatively enhanced in comparison to estradiol, in which the enzymatic makeup is aimed at an inactivation of estradiol. This applies for estrogen effects in the human uterus, especially if estrogen effects are greatly reduced under progesterone or gestagen dominance.

It can be shown that estriol can also trigger estrogen effects in the uterus under these conditions. The substances according to the invention are therefore especially suitable to be used in connection with gestagens for control of uterine bleeding behavior, e.g., in so-called “combined oral contraceptive agents” or gestagen-containing HRT products.

Subjects are pharmaceutical agents for ERT (estrogen replacement therapy) that contain compounds of general formula (I) according to the invention in very low dosages, such as, e.g., in a dose of 0.05 to 1 mg/day p.o. and that do not exert undesirable effects on the uterus (proliferation) and the liver (clotting factors or angiotensinogen).

Subjects are also pharmaceutical agents for ERT that contain compounds of general formula (I) according to the invention or salts thereof in very low dosages, such as, e.g., in a dose of 0.05 to 3 mg/day p.o., in combination with a gestagen. The gestagen can be progesterone, norethisterone, dienogest, cyproterone acetate, chlormadinone acetate, drospirenone, medroxy progesterone acetate, levonorgestrel, gestodene or another gestagen that is suitable for HRT. This can be used in the usual regimen and dosages, e.g., “continuous combined” or in variants of intermittent and sequential mode of administration.

Subjects are also pharmaceutical agents for contraception, the compounds of general formula (I) according to the invention or salts thereof in very low dosage, such as, e.g., in a dose of 0.05 to 3 mg/day p.o., in combination with a gestagen in the regimen that is common for oral contraceptive agents.

These pharmaceutical compositions and pharmaceutical agents can be used preferably for oral administration, but also for rectal, vaginal, subcutaneous, percutaneous, intravenous, transdermal or intramuscular administration. In addition to commonly used adjuvants, vehicles and/or diluents, they contain at least one compound of general formula I or salts thereof.

The pharmaceutical agents of the invention are produced with the commonly used solid or liquid vehicles or diluents and the commonly used pharmaceutical-technical adjuvants corresponding to the desired type of administration with a suitable dosage in a known way. The preferred preparations exist in a form for dispensing that is suitable for oral administration. Such forms for dispensing are, for example, tablets, film tablets, coated tablets, capsules, pills, powders, solutions or suspensions or else depot forms.

Of course, parenteral preparations such as injection solutions are also considered. In addition, for example, suppositories and agents for vaginal administration can also be mentioned as preparations.

Corresponding tablets can be obtained by, for example, mixing active ingredient with known adjuvants, for example inert diluents such as dextrose, sugar, sorbitol, mannitol, polyvinylpyrrolidone, explosives such as corn starch or alginic acid, binders such as starch or gelatins, lubricants such as magnesium stearate or talc and/or agents for achieving a depot effect such as carboxylpolymethylene, carboxylmethyl cellulose, cellulose acetate phthalate or polyvinyl acetate. The tablets can also consist of several layers.

Coating cores, which are produced analogously to the tablets, with agents that are commonly used in tablet coatings, for example polyvinyl pyrrolidone or shellac, gum Arabic, talc, titanium oxide or sugar, can accordingly produce coated tablets. In this case, the shell of the coated tablet can also consist of several layers, whereby the adjuvants that are mentioned above in the tablets can be used.

Solutions or suspensions with the compounds of general formula I according to the invention can contain additional taste-improving agents such as saccharine, cyclamate or sugar, as well as, e.g., flavoring substances such as vanilla or orange extract. In addition, they can contain suspending adjuvants such as sodium carboxy methyl cellulose or preservatives, such as p-hydroxybenzoates.

The capsules that contain compounds of general formula I can be produced by, for example, the compound(s) of general formula I being mixed with an inert vehicle such as lactose or sorbitol and encapsulated in gelatin capsules.

Suitable suppositories can be produced by, for example, mixing with vehicles that are provided for this purpose, such as neutral fats or polyethylene glycol or derivatives thereof.

The estriol and estetrol prodrugs according to the invention can be synthesized according to the examples below, whereby the latter are used for a more detailed explanation without limiting the invention.

General Synthesis Instructions

For the production of the compounds of general formulas IIA-D, known steroidal parent substances can be used. The following steroidal parent substances can be used, for example: estrone, estriol, and estetrol. The functional groups optionally can be protected according to methods that are known to one skilled in the art or can be converted into corresponding functionalities. Keto groups can thus be reduced into hydroxy compounds according to methods that are known to one skilled in the art and can be converted into enone and enol compounds. Double bonds can be converted into dihydroxy compounds according to methods that are known to one skilled in the art.

A) Coupling of the Steroidal Compound to Group Z

Variant I

Reaction with Sulfamoylphenylcarboxylic Acids

An estrogen is dissolved in a base, such as, e.g., pyridine. Corresponding amounts of a sulfamoylphenylcarboxylic acid are added to the solution, then an acid, such as, e.g., p-toluenesulfonic acid, and finally a carbodiimide, such as, e.g., dicyclohexylcarbodiimide, are added. The reaction mixture is stirred until the reaction is completed. Then, water is added, and it is acidified with an acid, such as, e.g., 10% HCl. The precipitate is filtered off, washed with water and NaHCO₃ solution and dried. The residue is extracted with an organic solvent, such as, e.g., ethyl acetate, the organic phase is washed and dried with a desiccant, such as, e.g., MgSO₄. After filtration, it is concentrated by evaporation and chromatographed on silica gel. Corresponding estrogen sulfamoyl benzoates are obtained.

Variant 2

Reaction with Sulfamoylphenylcarboxylic Acid Chlorides

An estrogen is dissolved in a base, such as, e.g., pyridine. The corresponding amount of a sulfamoylphenylcarboxylic acid chloride is added to the solution. The reaction mixture is stirred until the reaction is completed. Then, water is added, and it is acidified with an acid, such as, e.g., 10% HCl. It is extracted with an organic solvent, such as, e.g., ethyl acetate, the organic phase is washed, and it is dried with a desiccant, such as, e.g., MgSO₄. After filtration, it is concentrated by evaporation and chromatographed on silica gel. Corresponding estrogen sulfamoyl benzoates are obtained.

Variant 3

Reaction with Chlorosulfonylphenylcarboxylic Acid Chlorides

An estrogen is dissolved in a base, such as, e.g., pyridine, and an organic solvent, such as, e.g., chloroform, and cooled. The corresponding amount of a chlorosulfonylphenyl-carboxylic acid chloride is added to the solution. The reaction mixture is stirred at room temperature until the reaction is completed. Then, the reaction mixture is stirred into concentrated ammonia solution. The mixture is concentrated by evaporation and acidified with an acid, such as, e.g., 10% HCl. The precipitate is suctioned off, washed with water, dried and chromatographed on silica gel. Corresponding estrogen sulfamoyl benzoates are obtained.

Variant 4

Reaction with 2-Sulfophenylcarboxylic Acid-Cyclo-Anhydride

An estrogen is dissolved in an organic solvent, such as, e.g., chloroform. After 2-sulfophenylcarboxylic acid-cyclo-anhydride is added, it is stirred at elevated temperatures under a cover gas. Then, it is cooled and mixed with a concentrated ammonia solution, such as, e.g., methanolic ammonia solution. The solvent is distilled off, and the residue is chromatographed on silica gel. 2′-Sulfophenylcarboxylic acid ester-ammonium salts of corresponding estrogens, which are dissolved under a cover gas in an organic solvent, such as, e.g., CHCl₃, are obtained. A corresponding amount of a chlorinating agent, such as, e.g., PCl₅ or SOCl₂, is added in portions. The reaction mixture is stirred optionally at higher temperatures and then added briefly in concentrated NH₃ solution. The mixture is concentrated by evaporation, the precipitated substance is filtered off, washed with water, dried and chromatographed on silica gel. 2′-Sulfamoylphenylcarboxylic acid ester of corresponding estrogens is obtained.

Variant 5

Reaction to Form Sulfamides (NH₂SO₂NH—)

The reaction to form the sulfamides according to the invention is carried out according to methods that are known to one skilled in the art for their production starting from corresponding amines by means of sulfamide, sulfamoyl chloride or aminosulfonyl isocyanate (P. O. Burke et al., J. Chem. Soc. Perk. Trans 2, 1984, 1851; M. Preiss et al. Chem. Ber., 1978, 1915: C.-H. Lee et al., J. Org. Chem., 1990, 6104).

For example, a corresponding amino benzoate in an organic solvent, such as, e.g., toluene, is reacted in the presence of a base, such as, e.g., NEt₃, with sulfamoyl chloride at temperatures of 20-60° C. The reaction mixture is stirred until the reaction is completed. Then, water is added, the precipitate is filtered off, washed with water and NaHCO₃ solution and dried. The substance is purified by chromatography on silica gel. Corresponding estrogen sulfamoyl amino benzoates are obtained.

B) Synthesis of Group Z

### 2-Chloro-4-sulfamoylbenzoic Acid

Stage 1

10 g of 2-chloro-toluene-4-sulfonic acid-Na-salt×H₂O is added to 40 ml of thionyl chloride. After 5 ml of DMF is added, it is refluxed for 6 hours. The cold reaction mixture is added to 200 g of ice. The precipitated substance is washed with water and dried. 2-Chloro-toluene-4-sulfonic acid chloride is obtained.

¹H-NMR (DMSO-d₆): 2.32 (s, 3H, Me), 7.32-7.58 (m (superimposed), 3H, CH)

Stage 2

8 g of 2-chloro-toluene-4-sulfonic acid chloride is dissolved in 25 ml of CHCl₃ and slowly stirred into 100 ml of concentrated NH₃ solution. After 10 minutes of stirring at room temperature, the solution is concentrated by evaporation to one-half of its original volume. The substance is suctioned off, washed with water and dried. 2-Chloro-4-sulfamoyltoluene is obtained.

¹H-NMR (DMSO-d₆): 2.39 (s, 3H, Me), 7.44 (s, 2H, NH₂), 7.55-7.83 (m (superimposed), 3H, CH)

Stage 3

1.67 g of 2-chloro-4-sulfamoyltoluene is introduced into 70 ml of water. After 5 g of KMnO₄ and 0.5 ml of saturated NaHCO₃ solution are added, it is refluxed for 2 hours. After 2 ml of MeOH is added, the manganese dioxide that is produced is filtered off, and the solution is concentrated by evaporation to one-half of its original volume. After acidification with 10% HCl, the solution is cooled for 8 hours until crystallization is completed. Then, it is suctioned off, washed with water and dried. 2-Chloro-4-sulfamoylbenzoic acid is obtained.

¹H-NMR (DMSO-d₆): 7.66 (s, 2H, NH₂), 7.80-8.02 (m (superimposed), 3H, CH), 13.86 (s, 1H, COOH)

### 5-Sulfamoylisophthalic Acid

Stage 1

20 g of 5-sulfoisophthalic acid-Na-salt is boiled in 80 ml of thionyl chloride with the addition of 5 ml of DMF for 5 hours. The cold reaction mixture is added to 500 g of ice, and the precipitated substance is suctioned off, washed with water and dried. 5-Chlorosulfonyl-isophthalic acid dichloride is obtained.

¹H-NMR (CDCl₃): 8.98 (s, 2H, H-4,6), 9.11 (s, 1H, H-2)

Stage 2

10 g of 5-chlorosulfonylisophthalic acid dichloride is stirred into 150 ml of NH₃ solution in small portions. The solution is concentrated by evaporation, the precipitated substance is filtered off, washed with water and dried. 5-Sulfamoylisophthalic acid diamide is obtained.

¹H-NMR (DMSO-d₆): 7.51, 7.67, 8.22 (6H, NH₂), 8.43 (s, 2H, H-4,6), 8.53 (s, 1H, H-2)

Stage 3

1 g of 5-sulfamoylisophthalic acid diamide is suspended in 1,4-dioxane. 5 ml of 10% HCl is added, and the reaction mixture is heated for 3 hours to about 90° C. Then, the reaction mixture is evaporated to the dry state. The residue is chromatographed on silica gel. 5-Sulfamoylisophthalic acid-monoamide and 5-sulfamoylisophthalic acid are obtained.

### 4-Chlorosulfonylbenzoic Acid Chloride

15 g of 4-sulfonobenzoic acid-K-salt is dissolved in 100 ml of saturated ammonia solution. The solution is concentrated by evaporation, and the salt is dried on P₂O₅. 5 g of the salt is dissolved in 20 ml of SOCl₂. 0.3 ml of DMF is added to the reaction mixture and refluxed for 2 hours. It is allowed to cool off, toluene is added thereto for crystallization, and it is filtered off. The product is washed with toluene and dried. 4-Chlorosulfonylbenzoic acid chloride, which is used for further reactions, is obtained.

### SYNTHESIS EXAMPLES

### Example 1

### 3,16α-Dihydroxyestra-1,3,5(10)-trien-17β-yl 4′-sulfamoylbenzoate (1)

Stage 1

450 mg of 3,16α-di(tert.-butyldimethylsilyloxy)estra-1,3,5(10)-17β-ol is dissolved in 7 ml of pyridine. After the addition of 1.0 g of p-sulfamoylbenzoic acid, 120 mg of p-toluene sulfonic acid and 1.0 g of dicyclohexylcarbodiimide (DCC), it is stirred for 48 hours at room temperature. Then, 20 ml of water and 50 ml of ethyl acetate are added. It is slightly acidified with 10% HCl (pH=5). The precipitate is filtered off and rewashed with ethyl acetate. The organic phase is separated with 10% NaHCO₃ solution and washed with saturated NaCl solution, dried on MgSO₄, filtered, concentrated by evaporation and chromatographed on silica gel. 3,16α-Di(tert.-butyldimethylsilyloxy)estra-1,3,5(10)-trien-17β-yl 4′-sulfamoylbenzoate (2) is obtained.

¹H-NMR (CDCl₃): 0.04 (s, 6H, 2SiMe), 0.22 (s, 6H, 2SiMe), 0.87 (s, 9H, tert.-C₄H₉), 0.93 (s, 3H, H-18), 1.01 (s, 9H, tert.-C₄H₉), 4.51 (m, 1H, H-16), 5.04 (s, 2H, NH₂), 5.17 (m, 1H, H-17).

Stage 2

### 3-Hydroxy-16α-tert.-butyldimethylsilyloxyestra-1,3,5(10)-17β-yl 4′-sulfamoylbenzoate (3)

450 mg of 3,16α-di(tert.-butyldimethylsilyloxy)estra-1,3,5(10)-17β-yl 4′-sulfamoylbenzoate is dissolved in 10 ml of THF. While being stirred, 300 mg of TBAF is added at room temperature. After 1 hour, 40 ml of water is stirred in, and then the organic mobile solvent is distilled off. The substance is filtered off, washed with water, dried and chromatographed on silica gel. 3-Hydroxy-16α-tert.-butyldimethylsilyloxyestra-1,3,5(10)-trien-17β-yl 4′-sulfamoylbenzoate is obtained.

Stage 3

### 3,16α-Dihydroxyestra-1,3,5(10)-17β-yl 4′-sulfamoylbenzoate (1)

600 mg of 3-hydroxy-16α-tert.-butyldimethylsilyloxyestra-1,3,5(10)-17β-yl 4′-sulfamoylbenzoate is dissolved in 30 ml of THF. While being stirred, 1.5 ml of HCl (32%) is added at room temperature. After 2 hours, 20 ml of saturated NaHCO₃ solution is stirred in, and then the organic mobile solvent is distilled off. The substance is filtered off, washed with water, dried and chromatographed on silica gel. 3,16α-Dihydroxyestra-1,3,5(10)-trien-17β-yl 4′-sulfamoylbenzoate is obtained.

¹H-NMR (DMSO-d₆): 0.85 (s, 3H, H-18), 4.32 (m, 1H, H-16), 4.92 (d, 1H, H-17), 5.04 (m, 1H, 16-OH), 7.57 (m, 2H, NH₂), 8.99 (s, 1H, 3-OH).

### Example 2

### 3,17β-Dihydroxyestra-1,3,5(10)-trien-16α-yl 4′-sulfamoylbenzoate (4)

The substance is obtained analogously to Example 1 starting from 3,17β-di(tert.-butyldimethylsilyloxy)estra-1,3,5(10)-trien-16α-ol with the intermediate products 3,17β-di(tert.-butyldimethylsilyloxy)estra-1,3,5(10)-trien-16α-yl 4′-sulfamoylbenzoate (5) and 3-hydroxy-17β-tert.-butyldimethylsilyloxyestra-1,3,5(10)-trien-16α-yl 4′-sulfamoylbenzoate (6).

### 3,17β-Di(tert.-butyldimethylsilyloxy)estra-1,3,5(10)-trien-16α-yl 4′-sulfamoylbenzoate (5)

¹H-NMR (CDCl₃): -0.04 (s, 3H, SiMe), 0.09 (s, 3H, SiMe), 0.18 (s, 6H, 2SiMe), 0.86 (s, 9H, tert.-C₄H₉), 0.87 (s, 3H, H-18), 0.97 (s, 9H, tert.-C₄H₉), 3.94 (d, 1H, H-17), 5.09 (s, 2H, NH₂), 5.18 (m, 1H, H-16).

### 3,17β-Dihydroxyestra-1,3,5(10)-trien-16α-yl 4′-sulfamoylbenzoate (4)

¹H-NMR (DMSO-d₆): 0.78 (s, 3H, H-18), 3.81 (d, 1H, H-17), 5.09 (m, 1H, H-16), 5.20 (m, 1H, 17-OH), 7.55 (m, 2H, NH₂), 9.00 (s, 1H, 3-OH).

### Example 3

### 3,16α-Dihydroxyestra-1,3,5(10)-trien-17β-yl 3′-sulfamoylbenzoate (7)

Stage 1

### 3,16α-Di(tert.-butyldimethylsilyloxy)estra-1,3,5(10)-trien-17β-yl 3′-sulfamoylbenzoate (8)

1.5 g of 3,16α-di(tert.-butyldimethylsilyloxy)estra-1,3,5(10)-17β-ol is dissolved in 3 ml of pyridine and 3 ml of CHCl₃. 1.5 ml of 3-chlorosulfonylbenzoic acid chloride is added to the reaction mixture at −20° C. while being stirred. Then, it is heated to room temperature and stirred for 15 minutes. The reaction solution is added in 25 ml of concentrated NH₃ solution and stirred for 15 minutes. Then, the organic mobile solvent is distilled off. It is slightly acidified (pH=5) with 10% hydrochloric acid. The precipitated substance is suctioned off, washed with 10% NaHCO₃ solution and water and then dried. 3,16α-Di(tert.-butyldimethylsilyloxy)estra-1,3,5(10)-17β-yl 3′-sulfamoylbenzoate is obtained.

¹H-NMR (CDCl₃): −0.03 (s, 3H, SiMe), 0.02 (s, 3H, SiMe), 0.18 (s, 6H, 2SiMe), 0.84 (s, 9H, tert.-C₄H₉), 0.89 (s, 3H, H-18), 0.97 (s, 9H, tert.-C₄H₉), 4.48 (m, 1H, H-16), 4.71 (s, 2H, NH₂), 5.12 (m, 1H, H-17).

Stage 2

### 3-Hydroxy-16α-tert.-butyldimethylsilyloxyestra-1,3,5(10)-17β-yl 3′-sulfamoylbenzoate (9)

500 mg of 3,16α-di(tert.-butyldimethylsilyloxy)estra-1,3,5(10)-17β-yl 3′-sulfamoylbenzoate is dissolved in 10 ml of THF. 500 mg of TBAF is added while being stirred at room temperature. After 1 hour, 40 ml of water is stirred in, and then the organic mobile solvent is distilled off. The substance is filtered off, washed with water, dried and chromatographed on silica gel. 3-Hydroxy-16α-tert.-butyldimethylsilyloxyestra-1,3,5(10)-17β-yl 3′-sulfamoylbenzoate is obtained.

Stage 3

### 3,16α-Dihydroxyestra-1,3,5(10)-17β-yl 3′-sulfamoylbenzoate (7)

850 mg of 3-hydroxy-16α-tert.-butyldimethylsilyloxyestra-1,3,5(10)-17β-yl 3′-sulfamoylbenzoate is dissolved in 35 ml of THF. 1.5 ml of HCl (32%) is added at room temperature while being stirred. After 2 hours, 20 ml of saturated NaHCO₃ solution is stirred in, and then the organic mobile solvent is distilled off. The substance is filtered off, washed with water, dried and chromatographed on silica gel. 3,16α-Dihydroxyestra-1,3,5(10)-17β-yl 3′-sulfamoylbenzoate is obtained.

¹H-NMR (DMSO-d₆): 0.86 (s, 3H, H-18), 4.32 (m, 1H, H-16), 4.94 (d, 1H, H-17), 5.07 (d, 1H, 16-OH), 7.56 (m, 2H, NH₂), 9.00 (s, 1H, 3-OH).

### Example 4

### 3,17β-Dihydroxyestra-1,3,5(10)-trien-16α-yl 3′-sulfamoylbenzoate (10)

The substance is obtained analogously to Example 3 starting from 3,17β-di(tert.-butyldimethylsilyloxy)estra-1,3,5(10)-trien-16α-ol with the intermediate products 3,17β-di(tert.-butyldimethylsilyloxy)estra-1,3,5(10)-trien-16α-yl 3′-sulfamoylbenzoate (11) and 3-hydroxy-17β-tert.-butyldimethylsilyloxyestra-1,3,5(10)-trien-16α-yl 3′-sulfamoylbenzoate (12).

### 3,17β-Di(tert.-butyldimethylsilyloxy)estra-1,3,5(10)-trien-16α-yl 3′-sulfamoylbenzoate (11)

¹H-NMR (CDCl₃): −0.03 (s, 3H, SiMe), 0.09 (s, 3H, SiMe), 0.18 (s, 6H, 2SiMe), 0.86 (s, 9H, tert.-C₄H₉), 0.87 (s, 3H, H-18), 0.97 (s, 9H, tert.-C₄H₉), 3.95 (d, 1H, H-17), 5.02 (s, 2H, NH₂), 5.20 (m, 1H, H-16).

### 3,17β-Dihydroxyestra-1,3,5(10)-trien-16α-yl 3′-sulfamoylbenzoate (10)

¹H-NMR (DMSO-d₆): 0.79 (s, 3H, H-18), 3.82 (m, 1H, H-17), 5.09 (m, 1H, H-16), 5.20 (d, 1H, 17-OH), 7.55 (m, 2H, NH₂), 8.99 (s, 1H, 3-OH).

### Example 5

### 16α, 17β-Dihydroxyestra-1,3,5(10)-trien-3-yl 4′-sulfamoylbenzoate (13)

0.4 g of estriol is dissolved in 7 ml of pyridine. After 0.8 g of 4-sulfamoylbenzoic acid and 0.8 g of dicyclohexylcarbodiimide (DCC) are added, it is stirred for 1 hour at room temperature. Then, it is acidified with 10% HCl (pH=2), and 8 ml of water is added. The precipitate is filtered off and washed with 10% NaHCO₃ solution and water. The product that is obtained is chromatographed on silica gel. 16α,17β-Dihydroxyestra-1,3,5(10)-3-yl 4′-sulfamoylbenzoate is obtained.

¹H-NMR (DMSO-d₆): 0.69 (s, 3H, H-18), 3.32 (m, 1H, H-17), 3.85 (m, 1H, H-16), 7.62 (s, 2H, NH₂).

### LITERATURE

(1) Katzenellenbogen, B. S. and Korach, K. S. (1997) A New Actor in the Estrogen Receptor Drama—Enter ERβ. Endocrin. 138, 861-62/Kuiper, G. G. and Gustafsson, J. A. (1997) The Novel Estrogen Receptor-Beta Subtype: Potential Role in the Cell- and Promoter-Specific Actions of Estrogens and Antiestrogens. FEBS Lett. 410, 87-90
(2) Siiteri, P. K. and MacDonald, P. (1966) Placental Estrogen Biosynthesis During Human Pregnancy. J. Clin. Endocrinol. Metab. 26, 750/Brown, J. B. (1955) A Chemical Method for the Determination of Estriol, Estrone and Estradiol in Human Urine. Biochem. J. 60, 185/Buster, J. E.; Sakahini, J.; Killam, A. and Scragg, W. H. (1976) Late Pregnancy Rise in Estriol Concentration. Am. J. Obstet Gynecol 125, 672
(3) Svanberg, L. (1982) Effects of Estrogen Deficiency in Women Castrated When Young. Acta. Obstet. Gynecol. Scand. Suppl. 106, 11-15
(4) Christiansen, C. (1994) Postmenopausal Bone Loss and the Risk of Osteoporosis. Osteoporosis Int. 4, Suppl. 1, 47-51
(5) Cummings, S. R.; Browner, W. S.; Bauer, D.; Stone, K.; Ensrud, K.; Jamal, S. and Ettinger, B. (1998), Endogenous Hormones and the Risk of Hip and Vertebral Fractures Among Older Women. N. Engl. J. Med. 339, 733-38
(6) Parrish, H. M.; Carr, C. A.; Hall, D. G. and King, T. M. (1967) Time Interval from Castration in Premenopausal Women to Development of Excessive Coronary Atherosclerosis. Am J. Obstet. Gynecol 99, 155-162/Svanberg, L. (1982) Effects of Estrogen Deficiency in Women Castrated When Young. Acta. Obstet. Gynecol. Scand. Suppl. 106, 11-15/Witteman, J. C. M.; Grobbee, D. E.; Kok, F. J.; Hofmann, A. and Valkenburg, H. A. (1989) Increased Risk of Atherosclerosis in Women after the Menopause. Br. Med. J. 298, 642-44
(7) Falconer, C.; Ekman-Ordeberg, G.; Ulmsten, U.; Westergren-Thorsson, G.; Barchan, K. and Malmström, A. (1996) Changes in Paraurethral Connective Tissue at Menopause are Counteracted by Estrogen. Maturitas 24, 197-204
(8) Gentz, T.; Eiletz, J.; Kreuzer, G.; Pollow, K. and Schmidt-Gollwitzer, N I. (1980) Endocrine Regulation of the Endometrium throughout the Menstrual Cycle. Geburtshilfe Frauenheilkd. 40(11), 990-99
(9) Krattenmacher, R.; Knauthe, R.; Parczyk, K.; Walker, A; Hilgenfeldt, U. and Fritzemeier, K.-H. (1994), Estrogen Action on Hepatic Synthesis of Angiotensinogen and IGF-I: Direct and Indirect Estrogen Effects. J. Steroid. Biochem. Mol. Biol. 48, 207-14/Mashchak, C. A.; Lobo, R. A.; Dozono-Takano, R.; Eggena, P.; Nakamura, R. M.; Brenner, P. F. and Mishell, D. R., Jr. (1982), Comparison of Pharmacodynamic Properties of Various Estrogen Formulations. Am. J. Obstet. Gynecol. 144, 511-18/O'Sullivan, A. J. and Ho, K. K. Y. (1995), A Comparison of the Effects of Oral and Transdermal Estrogen Replacement on Insulin Sensitivity in Postmenopausal Women. J. Clin. Endocrinol. Metab. 80, 1783-8
(10) Goldzieher, J. W. (1990), Selected Aspects of the Pharmacokinetics and Metabolism of Ethinyl Estrogens and their Clinical Implications. Am. J. Obstet. Gynecol. 163, 318-22/Mandel, F. P.; Geola, F. L.; Lu, J. K. H.; Eggena, P.; Sambhi, M. P.; Hershman, J. M. and Judd, H. L. (1982), Biologic Effects of Various Doses of Ethinyl Estradiol in Postmenopausal Women. Obstet. Gynecol. 59, 673-9
(11) Helmer, O. M. and Griffith, R. S. (1952), The Effect of the Administration of Estrogens on the Renin Substrate (Hypertensinogen) Content on Rat Plasma. Endocrinology 51, 421-26/Krattenmacher, R.; Knauthe, R.; Parczyk, K.; Walker, A.; Hilgenfeldt, U. and Fritzemeier, K.-H. (1994), Estrogen Action on Hepatic Synthesis of Angiotensinogen and IGF-I: Direct and Indirect Estrogen Effects. J. Steroid. Biochem. Mol. Biol. 48, 207-14/Oelkers, W. K. H. (1996), Effects of Estrogens and Progestagens on the Renin-Aldosterone System and Blood Pressure. Steroids 61, 166-71
(12) Span, J. P. T.; Pieters, G. F. F. M.; Sweep, C. G. J.; Hermus, A. R. M. M. and Smals, A. G. H. (2000), Gender Difference in Insulin-like Growth Factor I Response to Growth Hormone (GH) Treatment in GH-Deficient Adults: Role of Sex Hormone Replacement. J. Clin. Endocrinol. Metab. 85, 1121-5/Kelly, J. J.; Rajkovic, I. A.; O'Sullivan, A. J.; Semia, C. and Ho, K. K. Y. (1993), Effects of Different Oral Estrogen Formulations on Insulin-like Growth Factor-I, Growth Hormone and Growth Hormone-Binding Protein in Post-Menopausal Women. Clin. Endocrinol. 39, 561-67
(13) Mashchak, C. A.; Lobo, R. A.; Dozono-Takano, R.; Eggena, P.; Nakamura, R. M.; Brenner, P. F. and Mishell, D. R., Jr. (1982), Comparison of Pharmacodynamic Properties of Various Estrogen Formulations. Am. J. Obstet. Gynecol. 144, 511-18
(14) C. Landolfi; M. Marchetti; G. Ciocci; and C. Milanese, Journal of Pharmacological and Toxicological Methods 38, 169-172 (1997).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosure of all applications, patents and publications, cited herein and of corresponding 102004025985.2 Application No. May 21, 2004 and U.S. Provisional Application Ser. No. 60/572,972, filed May 21, 2004 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Estriol and estetrol prodrugs of general formula (I), 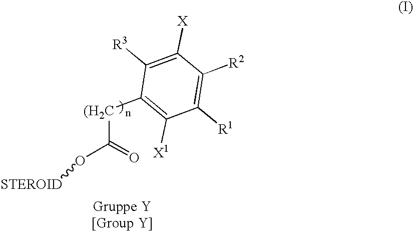
in which n is a number 0-4,
R¹ is a radical —SO₂NH₂ or —NHSO₂NH₂,
whereby R², R³ and X, X¹ stand for a hydrogen atom, a halogen atom,
a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and
R²⁰ in addition can mean a hydrogen, or
R² is a radical —SO₂NH₂ or —NHSO₂NH₂,
whereby R², R³ and X, X¹ stand for a hydrogen atom, a halogen atom,
a nitrile group, a nitro group, a C₁₋₅-alkyl group, a CₚF₂ₚ₊₁ group with p=1-3, a group OC(O)R²⁰, COOR²⁰, OR²⁰, C(O)NHR²⁰ or OC(O)NH—R²¹,
whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and
R²⁰ in addition can mean a hydrogen, or
R³ is a radical —SO₂NH₂ or —NHSO₂NH₂,
whereby R², R³ and X, X¹ stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
whereby R²⁰, R²¹ and R²² are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and
R²⁰ in addition can mean a hydrogen, and
STEROID stands for a steroidal ABCD-ring system of general partial formulas H A to II D 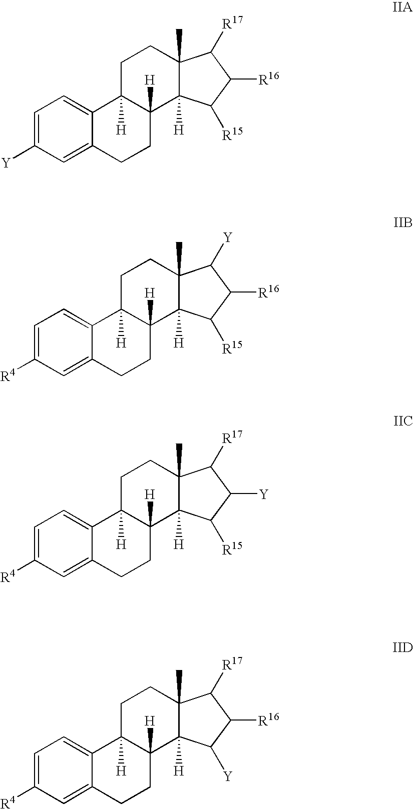
whereby
R⁴, R¹⁶, R¹⁷ can represent a hydroxy group, a tri(C₁-C₆-alkyl)silyloxy group, a group OC(O)—R²⁰; a C₂₋₅-heterocycloalkyloxy group, or a group Y and
R¹⁵ can represent a hydrogen atom, a hydroxy group, a tri(C₁-C₆-alkyl)silyloxy group, a group OC(O)R²⁰, a C₂₋₅-heterocycloalkyloxy group, or a group Y, and
their pharmaceutically acceptable salts.

2. Compounds according to claim 1, characterized in that n is 0, 1 or 2.

3. Compounds according to claim 1, wherein
R¹ represents a radical —SO₂NH₂ or —NHSO₂NH₂.

4. Compounds according to claim 3, wherein R¹ represents a group —SO₂NH₂.

5. Compounds according to claim 1, wherein either R¹, R² or R³ represents a group —SO₂NH₂.

6. Compounds according to claim 1, wherein R¹, R², R³, if the latter do not represent —SO₂NH₂ or —NHSO₂NH₂, as well as X and X¹, independently of one another, stand for a hydrogen, fluorine or chlorine atom, or a hydroxy or methoxy group.

7. Compounds according to claim 1, wherein R⁴, R¹⁶ and R¹⁷ in each case and independently of one another represent a hydroxy, trimethylsilyloxy, tert.-butyldimethylsilyloxy, benzoate, acetate, propionate, valerate, butciclate, or cyclopentylpropionate group or a group Y, and R¹⁵ represents a hydrogen atom.

8. Compounds according to claim 1, wherein STEROID stands for a steroidal ABCD-ring system of general partial formulas II B and II C.

9. Compounds according to claim 1, namely
1) 3,16α-Dihydroxyestra-1,3,5(10)-trien-17β-yl 3′-sulfamoylbenzoate (7),
2) 3,16α-Dihydroxyestra-1,3,5(10)trien-17β-yl 4′-sulfamoylbenzoate (1),
3) 3-tert.-butyldimethylsilyloxy-16α-hydroxyestra-1,3,5(10)-trien-17β-yl 3′sulfamoylbenzoate,
4) 3-tert.-butyldimethylsilyloxy-16α-hydroxyestra-1,3,5(10)-trien-17β-yl 4-sulfamoylbenzoate,
5) 3,17β-Dihydroxyestra-1,3,5(10)-trien-16α-yl 3′-sulfamoylbenzoate (10),
6) 3,17β-Dihydroxyestra-1,3,5(10)-trien-16α-yl 4′-sulfamoylbenzoate (4),
7) 3-tert.-butyldimethylsilyloxy-17β-hydroxyestra-1,3,5(10)-trien-16α-yl 3′-sulfamoylbenzoate,
8) 3-tert.-butyldimethylsilyloxy-17β-hydroxyestra-1,3,5(10)-trien-16α-yl 4′-sulfamoylbenzoate,
9) 3,16α-Dihydroxyestra-1,3,5(10)trien-17β-yl 2′-chloro-5′-sulfamoylbenzoate,
10) 16α, 17β-Dihydroxyestra-1,3,5(10)-trien-3-yl 4′-sulfamoylbenzoate (13),
11) 3,15α, 16α-Trihydroxyestra-1,3,5(10)-trien-17β-yl 3′-sulfamoylbenzoate,
12) 3,15α, 16α-Trihydroxyestra-1,3,5(10)-trien-17β-yl 4′-sulfamoylbenzoate,
13) 3,15α, 17β-Trihydroxyestra-1,3,5(10)-trien-16α-yl 3′-sulfamoylbenzoate,
14) 3,15α, 17β-Trihydroxyestra-1,3,5(10)-trien-16α-yl 4′-sulfamoylbenzoate,
15) 3,16α, 17β-Trihydroxyestra-1,3,5(10)-trien-15α-yl 3′-sulfamoylbenzoate,
16) 3,16α, 17β-Trihydroxyestra-1,3,5(10)-trien-15α-yl 4′-sulfamoylbenzoate,
17) 15α, 16α, 17β-Trihydroxyestra-1,3,5(10)-trien-3yl 3′-sulfamoylbenzoate,
18) 15α, 16α, 17β-Trihydroxyestra-1,3,5(10)-trien-3yl 4′-sulfamoylbenzoate.

10. Pharmaceutical compositions that contain at least one compound according to claim 1.

11. Pharmaceutical composition according to claim 10, wherein at least one additional steroidally active compound is included.

12. Pharmaceutical composition according to claim 11, wherein the additional steroidally active compound is a gestagen.

13. Pharmaceutical composition according to claim 12, wherein the gestagen is selected from the following group:
progesterone, norethisterone, dienogest, cyproterone acetate, chlormadinone acetate, drospirenone, medroxy progesterone acetate, levonorgestrel, and gestodene.

14. Use of the compounds according to claim 1 for the production of pharmaceutical agents for estrogen replacement therapy in women.

15. Use of the compounds according to claim 1 in birth control in women.

16. Uses of the compound according to claim 1 for the production of pharmaceutical agents for treating hormonally induced diseases in men and women.

17. Use according to claim 16 for the production of pharmaceutical agents for treatment of endometriosis, breast cancer, prostate cancer and hypogonadism.

18. Use of the compounds according to claim 1 for the production of pharmaceutical agents for treating diseases that can be positively influenced by the inhibition of carboanhydrase activity.

19. Process for the production of compounds of general formula (I) according to claim 1 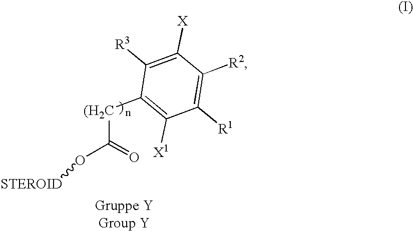
by reaction of estrogens with sulfamoylphenylcarboxylic acid or derivatives thereof or by reaction of corresponding compounds with sulfamide, sulfamoyl chloride or aminosulfonyl isocyanate.
